(19) United States
(12) Patent Application Publication
(10) Pub. No.: US 2025/0235085 A1
(43) Pub. Date: Jul. 24, 2025

(54) MODULAR ENDOSCOPE IMAGING GUIDEWIRE SYSTEMS AND METHODS

(71) Applicant: GYRUS ACMI, INC. D/B/A OLYMPUS SURGICAL TECHNOLOGIES AMERICA, Westborough, MA (US)

(72) Inventor: Nikhil M. Murdeshwar, Maple Grove, MN (US)

(21) Appl. No.: 18/703,629

(22) PCT Filed: Oct. 14, 2022
(86) PCT No.: PCT/US2022/078101

§ 371 Date: Apr. 22, 2024

Related U.S. Application Data
(60) Provisional application No. 63/262,899, filed on Oct. 22, 2021.
(60) Provisional application No. 63/267,616, filed on Feb. 7, 2022.

(51) Int. Cl. A61B 1/00; A61B 1/012; A61B 1/015; A61B 1/018; A61B 1/05; A61B 1/06; A61B 1/07; A61B 1/273

(52) U.S. Cl. CPC A61B 1/00105; A61B 1/00087; A61B 1/00096; A61B 1/0125; A61B 1/015; A61B 1/018; A61B 1/05; A61B 1/0607; A61B 1/07; A61B 1/273

(57) ABSTRACT

A modular endoscope system comprises an imaging guidewire comprising an elongate shaft extending from a proximal end to a distal end, an imaging device located proximate the distal end of the elongate shaft and a lighting element located proximate the distal end of the elongate shaft, and an intervention accessory configured to slide along the elongate shaft to provide a medical intervention. A method of providing a medical intervention to an internal anatomic location comprises inserting an imaging guidewire into an anatomic passageway, viewing target anatomy with imaging capabilities of the imaging guidewire, pushing an intervention accessory along the imaging guidewire to the target anatomy, and operating the intervention accessory to provide an intervention on the target anatomy.

### PRIORITY CLAIM

This application claims the benefit of priority to U.S. Provisional Patent Application Ser. No. 63/262,899, filed on Oct. 22, 2021, and U.S. Provisional Patent Application Ser. No. 63/267,616, filed on Feb. 7, 2022, the contents of which are incorporated herein by reference.

### TECHNICAL FIELD

The present disclosure relates generally to medical devices and instruments configured to provide diagnostic and treatment operations. More specifically, the present disclosure relates to medical device systems comprising elongate bodies that can be inserted into incisions or openings in anatomy of a patient and then advanced to reach locations deep within anatomic passageways of the patient where the diagnostic and treatment operations can be performed.

### BACKGROUND

Endoscopes can be used for one or more of 1) providing passage of other devices, e.g., therapeutic devices or tissue collection devices, toward various anatomical portions, and 2) imaging of such anatomical portions. Such anatomical portions can include the gastrointestinal tract (e.g., esophagus, stomach, duodenum, pancreaticobiliary duct, intestines, colon, and the like), the renal area (e.g., kidney(s), ureter, bladder, urethra) and other internal organs (e.g., reproductive systems, sinus cavities, submucosal regions, respiratory tract), and the like.

Conventional endoscopes can be involved in a variety of clinical procedures, including, for example, illuminating, imaging, detecting and diagnosing one or more disease states, providing fluid delivery (e.g., saline or other preparations via a fluid channel) toward an anatomical region, providing passage (e.g., via a working channel) of one or more therapeutic devices for sampling or treating an anatomical region, and providing suction passageways for collecting fluids (e.g., saline or other preparations) and the like.

In conventional endoscopy, the distal portion of the endoscope can be configured for supporting and orienting another instrument, such as via steering and the use of an elevator. In some systems, two endoscopes can be configured to work together with a first endoscope guiding a second endoscope inserted therein with the aid of an elevator that can turn the second endoscope relative to the first endoscope. Such systems can be helpful in guiding endoscopes to anatomic locations within the body that are difficult to reach. For example, some anatomic locations can only be accessed with an endoscope after passage through a circuitous path sometimes involving sharp turns between different anatomic passageways.

One example of an endoscopic procedure is called an Endoscopic Retrograde Cholangio-Pancreatography, hereinafter “ERCP” procedures. In an ERCP procedure, an “auxiliary scope” (also referred to as daughter scope or cholangioscope) can be attached and advanced through the working channel of a “main scope” (also referred to as mother scope or duodenoscope). Once the auxiliary scope has reached the desired location, various procedures can be performed, sometimes involving the use of an additional instrument or device. For example, a tissue retrieval device inserted through the auxiliary scope can be used to remove sample matter or a guidewire can be inserted through the auxiliary scope to place a stent.

### SUMMARY

The present inventor has recognized that problems to be solved with conventional medical devices, and in particular endoscopes and duodenoscopes used to diagnose and treat anatomy, include, among other things, 1) the difficulty in fully assessing anatomy and determining an intervention strategy until after a scope has been placed, such as a cholangioscope being positioned via a duodenoscope, 2) the diverging instruments used to perform different types of procedures at the same anatomic location, and 3) the reluctance of practitioners to use alternative medical device systems that have different capabilities than what the practitioners are accustomed to. In the case of 1), sometimes a tissue sampling procedure has to be performed in order to perform a biopsy before diseased tissue can be removed, thereby requiring a follow-up procedure. In the case of 2), sometimes a stone removal procedure can involve different accessories being used with the duodenoscope as compared to a tissue sampling procedure. In the case of 3), gastroenterologists can become pigeonholed into being able to perform a limited type and number of procedures if they cannot adapt to operating a wide variety of devices and instruments.

The present disclosure can help provide solutions to these and other problems by providing systems, devices and methods capable of performing a plurality of different procedures utilizing features that are familiar to a wide range of surgeons. The present disclosure includes various modular scope systems that provide surgeons with a wide variety of backgrounds a familiar starting point for performing a procedure using a first instrument having readably useable features. Thereafter, a second instrument can be used in conjunction with the first instrument to provide a desired interventional outcome, such as a diagnostic procedure, a tissue removal procedure or an implantation procedure, using technology and features specific to such procedures that are familiar to different surgeons. In examples, the first instrument can comprise an imaging guidewire and the second instrument can comprise one of a plurality of accessories configured to attach to or slide along the imaging guidewire. The imaging guidewire can have camera and lighting capabilities and a small diameter to facilitate navigation to a desired anatomic location. The camera capabilities can be used to assess the anatomy and then a shaft of the imaging guidewire can be used to guide a selected accessory to the anatomic location without needing independent guidance to perform the desired procedure.

In an example, a modular endoscope system can comprise an imaging guidewire comprising an elongate shaft extending from a proximal end to a distal end, an imaging device located proximate the distal end of the elongate shaft and a lighting element located proximate the distal end of the elongate shaft, and an intervention accessory configured to slide along the elongate shaft to provide a medical intervention.

In another example, a method of providing a medical intervention to an internal anatomic location can comprise inserting an imaging guidewire into an anatomic passageway, viewing target anatomy with imaging capabilities of the imaging guidewire, pushing an intervention accessory along the imaging guidewire to the target anatomy, and operating the intervention accessory to provide an intervention on the target anatomy.

### BRIEF DESCRIPTION OF DRAWINGS

FIG. 1 is a schematic diagram of a modular scope system comprising an imaging guidewire and accessories configured to ride along the imaging guidewire, the accessories comprising a concentrically mounted accessory and an under-mounted accessory.

FIG. 2A is a schematic diagram of the concentrically mounted accessory of FIG. 1 disposed about an exterior of the imaging guidewire.

FIG. 2B is a schematic diagram of the under-mounted accessory of FIG. 1 attached to a bottom portion of the imaging guidewire.

FIG. 3 is a schematic diagram of an endoscopy system comprising a duodenoscope and an imaging and control system comprising a control unit connected to the duodenoscope.

FIG. 4 is schematic diagram of the imaging and control system of FIG. 3 connected to the duodenoscope.

FIG. 5A is a schematic top view of a camera module including optical components for a side-viewing endoscope and an elevator mechanism.

FIG. 5B is an enlarged cross-sectional view taken along the plane 5B-5B of FIG. 5A showing the optical components.

FIG. 5C is an enlarged cross-sectional view taken along the plane 5C-5C of FIG. 5A showing the elevator mechanism.

FIG. 6 is a schematic diagram illustrating the duodenoscope of FIGS. 3-5C positioning the imaging guidewire of FIGS. 1-2B in a common bile duct of a duodenum.

FIGS. 7A and 7B are schematic views of a concentrically mounted accessory of FIGS. 1 and 2A comprising a stent mounted to the imaging guidewire in collapsed and expanded states, respectively.

FIG. 7C is a schematic view of the duodenum of FIG. 6 with the stent of FIGS. 7A and 7B inserted into the duodenal papilla of a common bile duct.

FIG. 8 is a close-up schematic view of a rail system used to connect the under-mounted accessory of FIGS. 1 and 2B comprising a working channel and auxiliary channels.

FIG. 9 is a schematic view of a modular scope system comprising an imaging guidewire attached to an accessory module having a fluid channel and an integrated laser fiber.

FIG. 10 is a schematic view of a modular scope system comprising an imaging guidewire attached to an accessory module having a fluid channel and a working channel into which is inserted a removal device.

FIG. 11 is a schematic view of a modular scope system comprising an imaging guidewire extending through accessory module having a fluid channel and a working channel into which is inserted a removal device.

### DETAILED DESCRIPTION

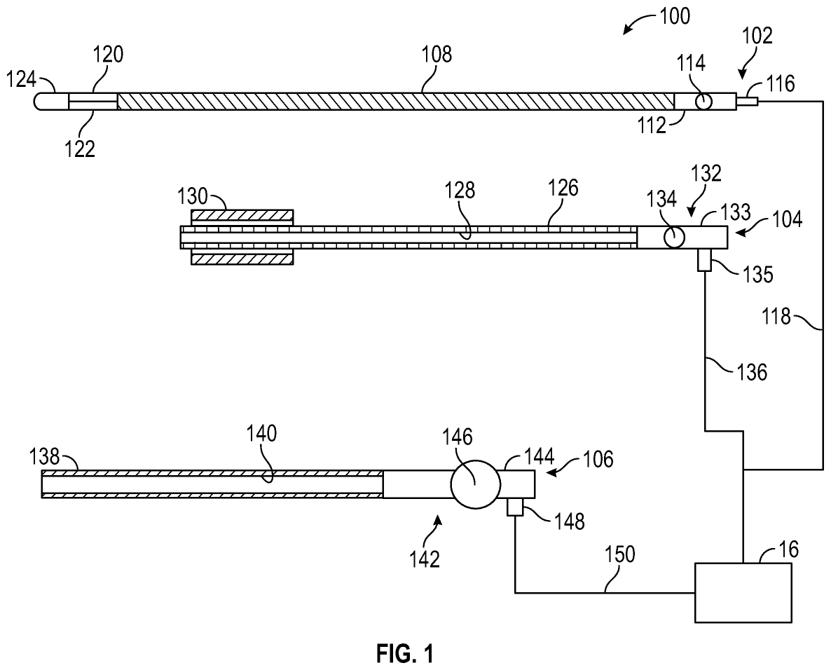

FIG. 1 is a schematic diagram of modular scope system 100 comprising imaging guidewire 102 and accessories configured to ride along imaging guidewire 102, the accessories comprising concentrically mounted accessory 104 and under-mounted accessory 106. FIG. 1 is not necessarily drawn to scale and may be exaggerated in certain aspects for illustrative purposes.

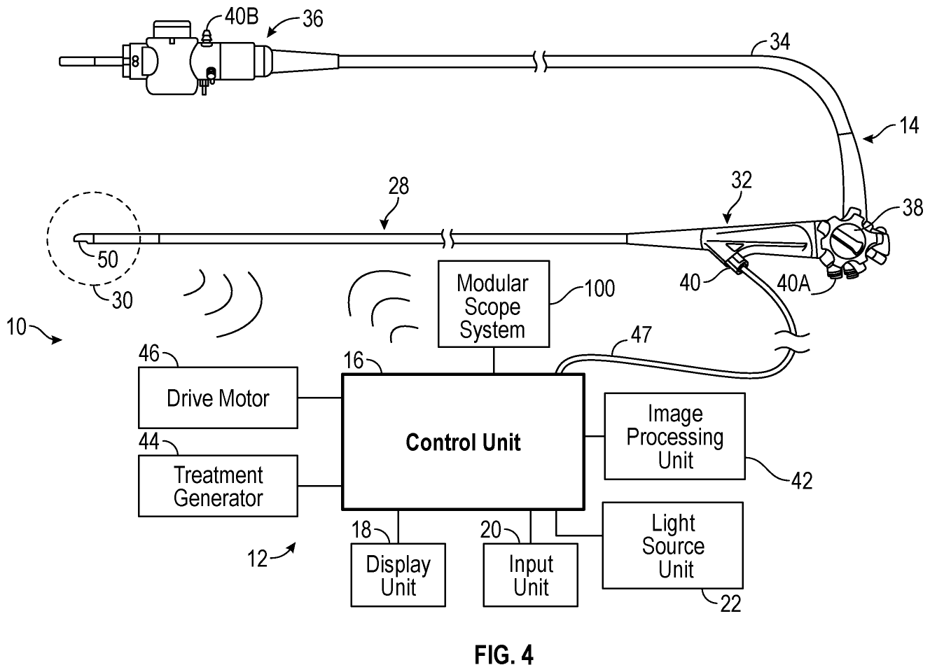

System 100 can comprise imaging guidewire 102, concentrically mounted accessory 104 and under-mounted accessory 106. Imaging guidewire 102 can comprise shaft 108 and control device 110, which can include grip 112, control knob 114 and coupler 116 that can connect to control unit 16 (FIG. 4) via cable 118. Imaging guidewire 102 can further comprise viewing module 119, comprising imaging device 120 and lighting device 122, and nosecone 124.

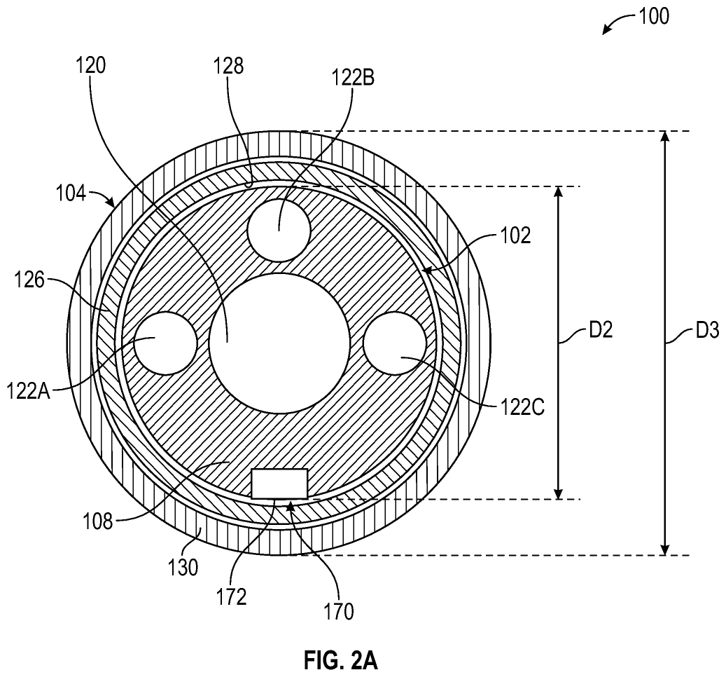

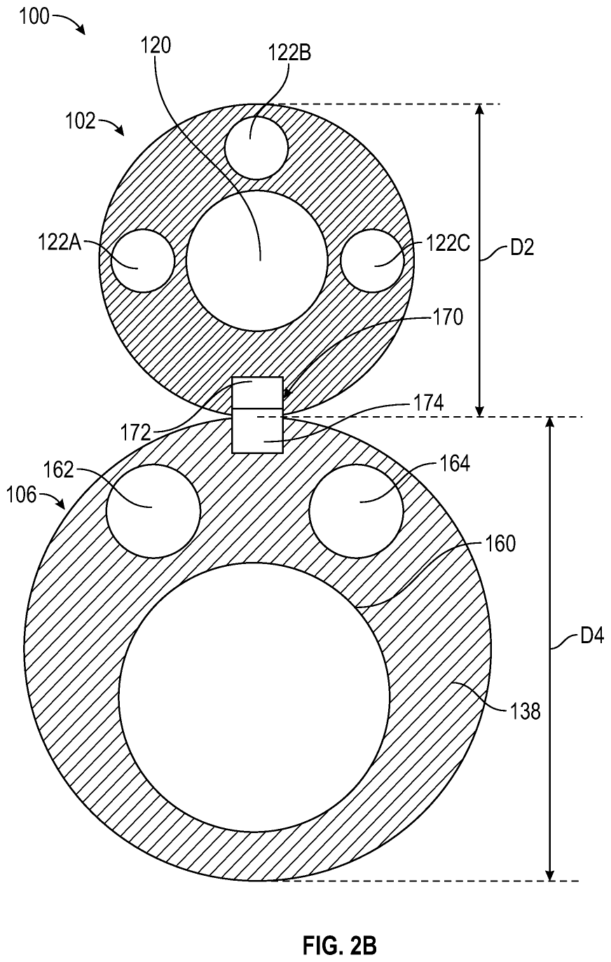

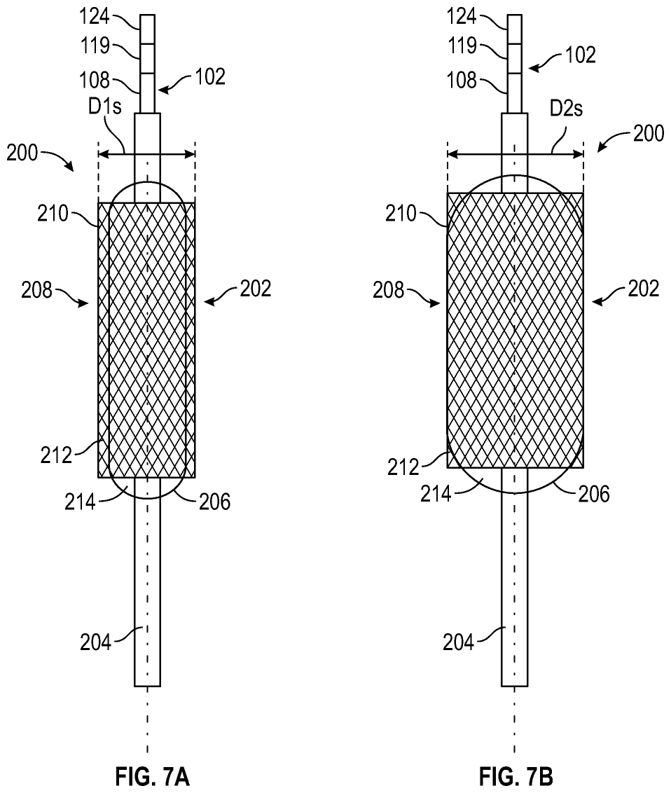

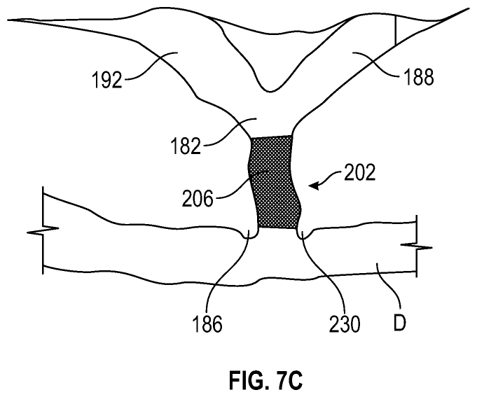

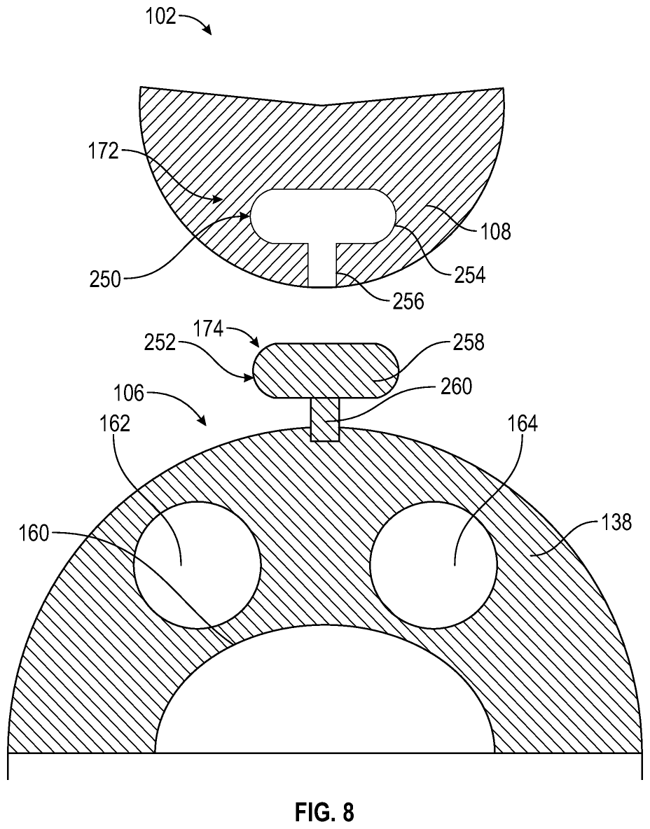

Concentrically mounted accessory 104, which is described in greater detail with reference to FIGS. 2A and 7A-7C, can comprise shaft 126 comprising lumen 128, deployable device 130 and control device 132. Control device 132 can comprise grip 133, control knob 134 and coupler 135 that can connect to control unit 16 via cable 136.

Under-mounted accessory 106, which is described in greater detail with reference to FIGS. 2B and 8-10, can comprise shaft 138 comprising lumen 140 and control device 142. Control device 142 can comprise grip 144, control knob 146 and coupler 148 that can connect to control unit 16 via cable 150.

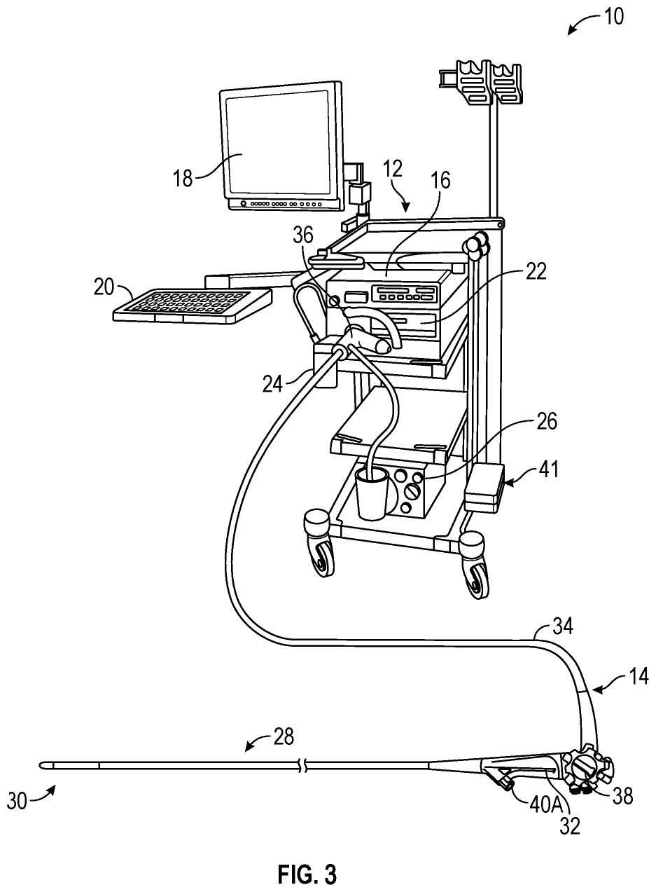

Control unit 16 can provide operational capabilities to system 100, such as power, intervention energization, irrigation fluid, suction, and the like. Control unit 16 is described in greater detail with reference to FIGS. 3 and 4.

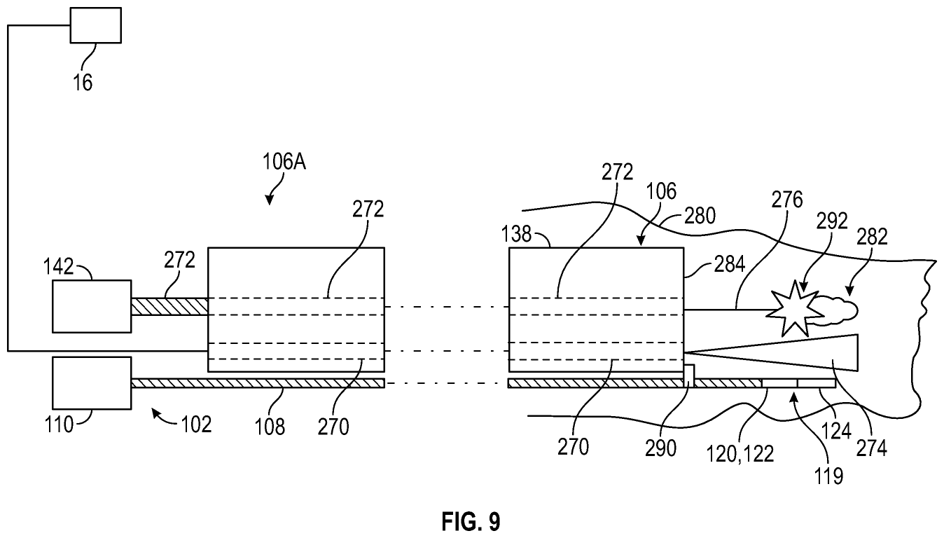

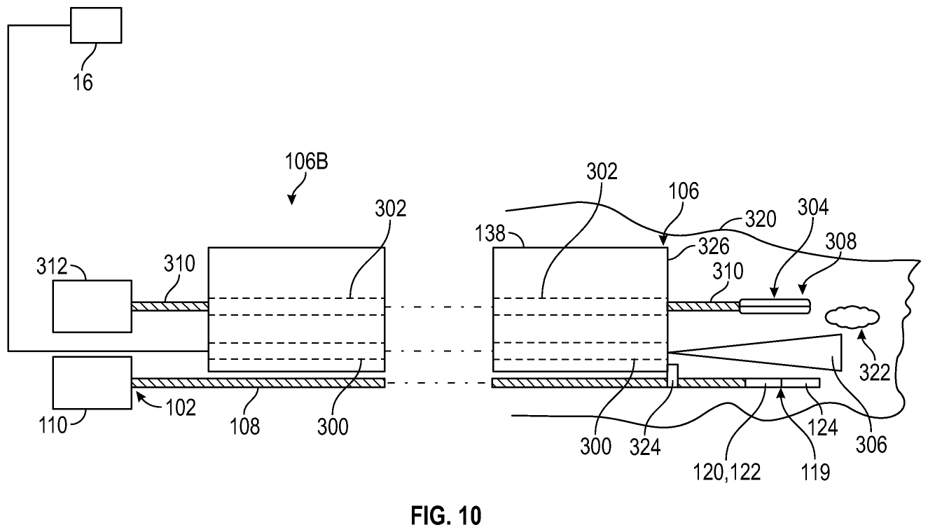

As is discussed in greater detail herein, imaging guidewire 102 can be guided to an anatomic site to facilitate diagnosing and assessing anatomic features at which an intervention, such as a sampling or treatment, can be performed. In examples, imaging guidewire 102 can be guided to the anatomic site via a duodenoscope, as discussed with reference to FIGS. 3-5C. Once the anatomic features are diagnosed, an accessory device can be guided to the anatomic site via imaging guidewire 102. As such a surgeon can decide intraoperatively a treatment plan that is beneficial for the patient or that is expedient for the surgeon. Accessory 104 can be slid over imaging guidewire 102 to the anatomic site. In examples, accessory 104 can comprise a stent delivery system and a stent. Accessory 106 can be slid alongside imaging guidewire 102 via attachment mechanism 170 (FIG. 2B), whether under, above or alongside. In examples, accessory 106 can comprise a body having an integrated stone fragmentation capability and an irrigation system, as shown in FIG. 9. In examples, accessory 106 can comprise a body including a working channel to guide another instrument, including tissue removers, tissue retrievers, forceps, electrohydraulic lithotripsy (EHL) probes, baskets, and the like, as shown in FIG. 10. As such, modular scope system 100 can reduce procedure complexity by providing imaging guidewire 102 that can be readily used by a diverse set of surgeons having different skill sets. Imaging guidewire 102 can then facilitate usage of a specific-needs accessory that can be employed using specialty skill sets of the different surgeons. Imaging guidewire 102 can remain in the patient while one or more accessories can be passively delivered to the anatomic site of the target anatomy using imaging guidewire 102, thereby reducing the number of times that individual instruments are actively guided, steered or navigated into the duodenum and common bile duct, or other anatomic locations, to perform various procedures.

Imaging guidewire 102 can be configured as an imaging device that can be steered and navigated to a desired anatomic location to view and evaluate the anatomy. Imaging guidewire 102 can be directed to the desired anatomic location using fluoroscopy, if desired. Shaft 108 of imaging guidewire 102 can include pull wires (not shown) that can be used to steer imaging guidewire 102. Control device 110 can be used to operate imaging guidewire 102, including the pull wires. For example, grip 112 can be grasped by an operator and control knob 114 can be rotated to pull on one or both of the pull wires to apply directionality to the shape of shaft 108.

In examples, shaft 108 can include a lumen (not illustrated) to allow for the passage of connecting elements, such as wires and cables, for imaging device 120 and lighting device 122. In examples, shaft 108 can comprise a sheath disposed about such wires and cables. In examples, viewing module 119 can include a wireless communication circuit including one or more transponders or beacons that can communicate using well-established wireless communication protocols, such as 3G, 4G, 5G, Bluetooth®, and wireless internet protocols such as 802.11 and WiFi. In advantageous aspects, Bluetooth can be used to achieve desirable data transfer rates and low power consumption rates.

Shaft 108 of imaging guidewire 102 can be flexible to facilitate insertion through various shaped anatomies. Shaft 108 can be made of suitable materials compliant enough to be directed by pull wires, but rigid enough to allow for insertion through anatomy. In examples, shaft 108 can be fabricated from various polymers. In the illustrated example, shaft 108 can comprise a circular cross-section centered on a central axis. In other examples, shaft 108 can have other cross-sectional profiles, such as rectilinear and polygonal. Control device 110 can have the same or a similar cross-sectional profile as shaft 108 so at allow other components, such as accessory 104, to fit onto shaft 108 from the proximal end. In examples, one or both of control device 110 can be detachable from shaft 108 and coupler 116 can be detachable from control device 110 to allow other components, such as accessory 104, to fit onto shaft 108 from the proximal end.

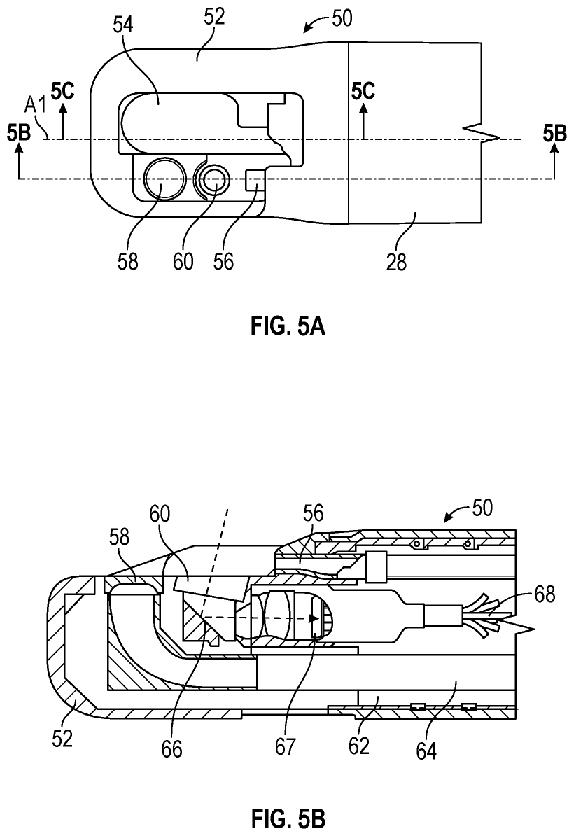

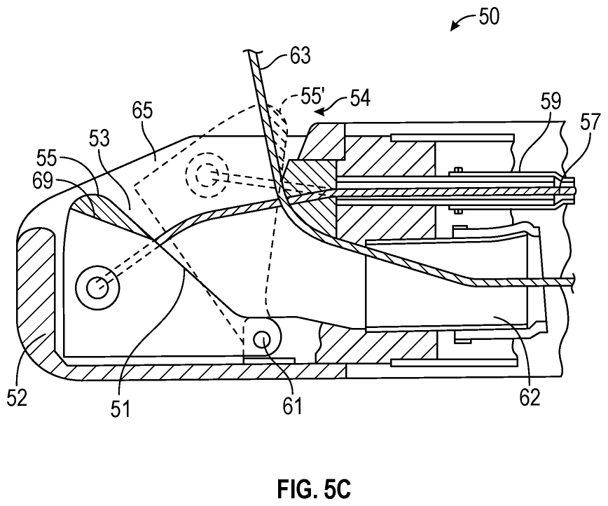

Control device 110 can additionally be used to operate imaging device 120 and lighting device 122. Control knob 114 or another component can comprise buttons, switches and the like to selectively power on and power off imaging device 120 and lighting device 122. In examples, the brightness of lighting device 122 can be adjusted. Imaging device 120 can comprise a camera, similar to what is described with reference to objective lens 60 of FIG. 5A. Lighting device 122 can be configured to emit light waves, similar to what is described with reference to lens 58 of FIG. 5A. Lighting device 122 can comprise a light-emitting fiber connected to a light generator in control device 110 or control unit 16 (FIG. 3). Viewing module 119 can also include a photosensitive element, such as a charge-coupled device (“CCD” sensor) or a complementary metal-oxide semiconductor (“CMOS”) sensor to, for example, obtain video images. In either example, imaging device 120 can be coupled (e.g., via wired or wireless connections) to image processing unit 42 (FIG. 4) to transmit signals from the photosensitive element representing images (e.g., video signals) to image processing unit 42, in turn to be displayed on a display such as output unit 18 (FIG. 3). In various examples, imaging and control system 12 (FIG. 3) and imaging device 120 can be configured to provide outputs at desired resolution (e.g., at least 480p, at least 720p, at least 1080p, at least 4K UHD, etc.) suitable for endoscopy procedures.

Nosecone 124 can comprise a cover positioned in front of viewing module 119 and can be attached thereto. Nosecone 124 be configured to shield viewing module 119 and prevent fluid from entering imaging guidewire 102. Nosecone 124 can additionally be tapered and have a rounded leading edge or tip to prevent damage to tissue and help push tissue out of the way of imaging guidewire during insertion. Nosecone 124 can be made of a soft, pliant material and can also be clear or transparent to allow light waves to pass to and from viewing module 119. Nosecone 124 can additionally be configured as a stop to prevent accessories from sliding off of imaging guidewire 102, such as by having a diameter slightly larger than imaging guidewire 102 or by closing off the end of a slide channel used to couple to an accessory.

In examples, imaging guidewire 102 can be configured similarly to a fully functional scope including steerability, guidance capability, imaging capability, and lighting capabilities, but without a functional capability, e.g., therapeutic and diagnostic capabilities. Functional capabilities for imaging guidewire 102 can be provided by different accessories, such as concentrically mounted accessory 104 and under-mounted accessory 106.

In examples, concentrically mounted accessory 104 can be configured to provide functional capabilities to imaging guidewire 102 as other conventional devices used with guidewires, such as catheters and stents and delivery systems for such implantable devices. Concentrically mounted accessory 104 can include shaft 126 and deployable device 130, which can be configured as a stent. Imaging guidewire 102 can be positioned within lumen 128 of shaft 126 and deployable device 130 can be positioned around shaft 126, as shown in FIG. 2A.

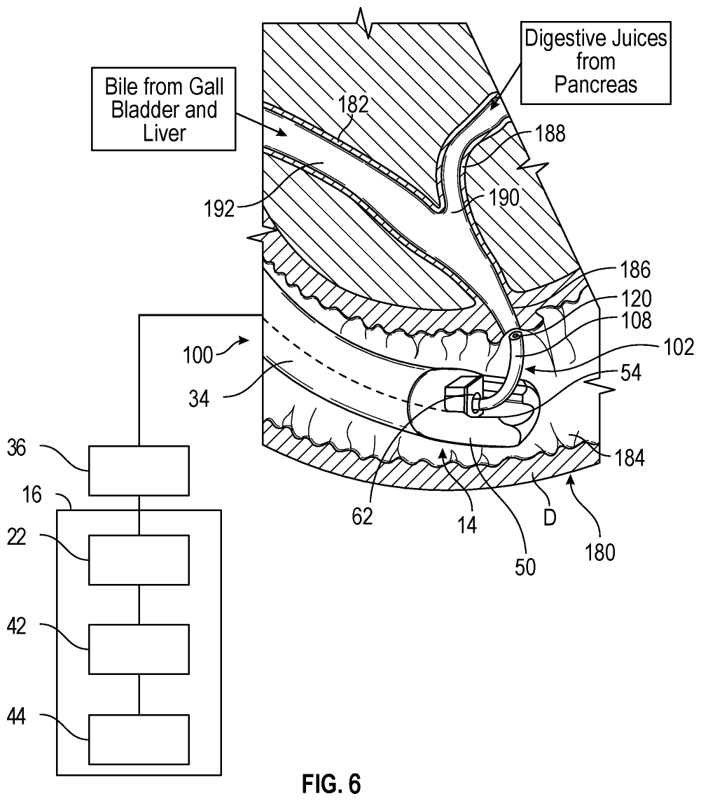

In examples, under-mounted accessory 106 can be configured to provide the complimentary functional capabilities to imaging guidewire 102 as a conventional endoscope, such as a cholangioscope. Under-mounted accessory 106 can include working channel 160, irrigation channel 162 and auxiliary channel 164, as shown in FIG. 2B. Imaging guidewire 102 can be sized to occupy less than the total area available within a working channel of a mother scope, such as a duodenoscope, to allow for the passage of an additional device through the mother scope simultaneously with imaging guidewire 102. The combined size of imaging guidewire 102 and one of accessory 104 and accessory 106 can be configured to fit within the working channel of a duodenoscope, e.g., endoscope 14 of FIGS. 3 and 4. In examples, the outer diameter of a duodenoscope, can be in in the range of approximately 10 mm to approximately 12.0 mm. In additional examples, the diameter of a working channel (“diameter D1”) of a duodenoscope, such as lumen 62 of FIG. 6, can be approximately 5.0 mm or smaller, as discussed below.

Shaft 108 of imaging guidewire 102 can have a second diameter. Diameter D2 can be as small as possible while still being able to provide sufficient strength and flexibility as a guide wire and connect to imaging devices that are able to obtain suitable images with sufficiently large fields of view, etc. In examples, D2 can be approximately 3.0 mm or smaller. In additional examples, D2 can be in the range of approximately 1.0 mm to approximately 0.8 mm or smaller, as discussed below. For comparison, existing cholangioscope designs typically have outer diameters of about 3.4 mm with typical, non-imaging guidewires having diameters of about 1.0 to 0.8 mm.

Concentrically mounted accessory 104 can have a third diameter D3 (FIG. 2A). The outer diameter D3 of concentrically mounted accessory 104 can be sized to fit within the working channel of a duodenoscope alongside shaft 108. Second diameter D2 can be in the range starting from just smaller than D1 to just larger than D2. In examples, diameter D3 can be in in the range of approximately 3.4 mm or smaller. Shaft 126 of concentrically mounted accessory 104 can be configured to freely slide around shaft 108 of imaging guidewire 102.

Under-mounted accessory 106 can have a fourth diameter D4 (FIG. 2B). The outer diameter D4 of under-mounted accessory 106 can be sized to fit within the working channel of a duodenoscope alongside shaft 108. Diameter D4 can be as large as the difference between D1 and D2. In examples, diameter D4 can be approximately 5.0 mm or smaller.

The sizes and dimensions of duodenoscope (e.g., scope 14 of FIG. 3), imaging guidewire 102, concentrically mounted accessory 104 and under-mounted accessory 106 can be selected or manufactured as part of a set or system configured to have compatibility as discussed herein. As such, the use of dedicated daughter devices within a mother device that are configured to perform specific tasks within the anatomy after insertion through the mother scope can be avoided or eliminated. For example, the use of a cholangioscope can be replaced by the use of imaging guidewire 102 and an accessory having the capabilities of a cholangioscope, including the scopes described herein that have the capabilities of a cholangioscope and a built-in lithotripter. Likewise, the use of a stent delivery system can be replaced by the use of imaging guidewire 102 and an accessory having the capabilities of a stent delivery system. Thus, imaging guidewire 102 can be widely adapted by different surgeons who use different daughter devices with a mother device to perform the same procedure, as well as different surgeons who perform different procedures with the same mother device. Thus, hospitals and facilities can reduce inventory and training required to provide wide ranging endoscopy procedures, particularly in the gastrointestinal area.

FIG. 2A is a schematic diagram of concentrically mounted accessory 104 of FIG. 1 disposed about an exterior of imaging guidewire 102. FIG. 2A can represent an end view of the distal end of concentrically mounted accessory 104. Imaging guidewire 102 can comprise shaft 108, imaging device 120 and lighting devices 122A, 122B and 122C. Concentrically mounted accessory 104 can comprise shaft 126 having lumen 128 and deployable device 130.

Imaging guidewire 102 can be configured to be inserted through anatomy to facilitate insertion of accessory 104 thereafter. Accessory 104 can be configured as any number of different systems that can be used to deliver a device via a guidewire. Shaft 126 can comprise a delivery device configured to carry deployable device 130. In examples, deployable device 130 can comprise a stent, medication delivery device, filter, valve, catheter (e.g., a balloon dilatation catheter) and the like.

In the illustrated example, shaft 126 can comprise a delivery device configured to carry deployable device 130 comprising a stent. Shaft 126 can comprise a tube having an inflatable portion over which deployable device 130 is positioned. Shaft 126 can be used to push deployable device 130 to the desired anatomic area over imaging guidewire 102. Deployable device 130 can comprise a stent comprising a mesh sleeve that can be switched from a collapsed configuration having a first small diameter to an expanded configuration having a second larger diameter, as is discussed with reference to FIGS. 7A-7C. Pressurized air or gas can be directed into lumen 128 to inflate the inflatable portion, thereby expanding deployable device 130 from the collapsed configuration to the expanded configuration. Control device 132 can be operated, such as via 134, to selectively control flow of a pressurizing medium through shaft 126 and into deployable device 130. Deployable device 130 can hold the form of the expanded configuration when the pressurizing medium is no longer provided. Thus, deployable device 130 can be used to open anatomic passages, such as Sphincter of Oddi 186 (FIG. 6) thereby making it easier to insert other medical devices and instruments.

Concentrically mounted accessory 104 can take advantage of the presence of lumen 128, as is incorporated into the existing designs of various annularly shaped devices, that can allow accessory 104 to be positioned over imaging guidewire 102. Additionally, the radial symmetry of shaft 126 and deployable device 130 can facilitate functionality between imaging guidewire 102 and a delivery device, such as a duodenoscope. Though described as concentrically mounted, accessory 104 need not be coaxial with imaging guidewire 102. Additionally, concentrically mounted accessory 104 or other accessories need not fully surround imaging guidewire 102 around the entire circumference of imaging guidewire 102. Furthermore, the outer perimeters of imaging guidewire 102 and accessory 104 need not be circular and the outer perimeter of imaging guidewire 102 and the shape of lumen 128 need not be complimentary.

Lumen 128 can allow concentrically mounted accessory 104 to be attached to imaging guidewire 102 in a slidable state without the use of separate attachment or coupling features. As discussed above, the diameter D2 of imaging guidewire 102 can be small to allow for passage of a wide variety of accessories thereover. The diameter D3 of concentrically mounted accessory 104 can be as large as desired to fit within anatomy or the working channel of another scope.

Imaging guidewire 102 can further comprise guidewire component 172 of attachment mechanism 170. Guidewire component 172 can be positioned on imaging guidewire 102 to facilitate attachment of other accessories to shaft 108 without having to rely on the accessory circumscribing or partially surrounding imaging guidewire 102, as is discussed in greater detail with reference to FIG. 2B. Guidewire component 172 can be located inside the outer perimeter or boundary of shaft 108 such that guidewire component 172 does not interfere with sliding of accessory 104 around imaging guidewire 102. Shaft 126 can be configured to slide past the distal end of imaging guidewire 102, past nosecone 124, thereby allowing deployable device to be placed within the anatomy and separated from imaging guidewire 102 within the anatomy.

FIG. 2B is a schematic diagram of under-mounted accessory 106 of FIG. 1 attached to a bottom portion of imaging guidewire 102. FIG. 2B can represent an end view of the distal end of under-mounted accessory 106. Imaging guidewire 102 can comprise shaft 108, imaging device 120 and lighting devices 122A-122C. Under-mounted accessory 106 can comprise shaft 138 comprising working channel 160, irrigation channel 162 and auxiliary channel 164. Imaging guidewire 102 and under-mounted accessory 106 can be attached via attachment mechanism 170, which can comprise guidewire component 172 and accessory component 174.

As is discussed with reference to FIGS. 9 and 10, under-mounted accessory 106 can be configured with a plurality of different capabilities to meet the needs and desires of different users and the requirements of different procedures. In the illustrated example of FIG. 2B, working channel 160 can comprise a hollow passage or lumen configured to receive another instrument or device, irrigation channel 162 can be connected to a source of fluid, such as saline, to dispense fluid into anatomy, and auxiliary channel 164 can be configured as desired to receive a fluid or another device. Additionally, one or more auxiliary channels 164 can be provided to incorporate steering or pull wires that can be connected to control device 142 to cause a turning or bending of shaft 138. Any one or more of working channel 160, irrigation channel 162 and auxiliary channel 164 can represent lumen 140 of FIG. 1.

FIG. 2B illustrates guidewire component 172 and accessory component 174 of attachment mechanism 170 schematically. Attachment mechanism 170 can comprise any device configured to radially attach accessory 106 to imaging guidewire 102, while additionally permitting axial movement therebetween. Attachment mechanism 170 can extend along the length of the axial interface between imaging guidewire 102 and accessory 106 or can be located intermittently along the axial interface. As discussed below, guidewire component 172 can comprise a slot and accessory component 174 can comprise a rail configured to ride in the slot. In examples, guidewire component 172 can include a stop that prevents accessory component 174 from sliding off the end of imaging guidewire 102. In examples, slots comprising guidewire component 172 can terminate short of the distal end of shaft 108 thereby being configured to allow accessory component 174 to abut the end of the slot to prevent the accessory from disengaging imaging guidewire 102 or sliding past viewing module 119. As mentioned, FIG. 2B is described with reference to a slot and rail configuration, but other devices can be used to maintain imaging guidewire 102 and under-mounted accessory 106 is an axially slidable relationship, such as loops, magnets, and other devices, while also permitting concentrically mounted accessory 104 to fit over imaging guidewire 102. For example, flexible straps can be attached to imaging guidewire to form loops that can receive under-mounted accessory 106, but that can fold into concentrically mounted accessory 104. Additionally, magnets can be positioned along the length of imaging guidewire 102 to interact with a metallic or magnetic strip on under-mounted accessory 106.

FIGS. 2A and 2B show lighting devices 122A-122C as comprising three separate devices positioned to surround imaging device 120. Guidewire component 172 can be located between lighting devices 122A and 122C to help keep the size of imaging guidewire 102 small. Likewise, steering or pull wires can be located between spaced apart lighting devices 122A-122C that can be connected to control device 110 to cause a turning or bending of shaft 108. However, any number of lighting devices can be used. In examples, one or more of lighting devices can be arranged to form a ring light with imaging device 120 in the center. In additional examples, imaging device 120 and one or more lighting devices can be disposed in other arrangements. For example, a single lighting device and imaging device 120 can be arranged in a side-by-side configuration. Additionally, multiple lighting devices can be arranged in side-by-side configurations. Lighting devices 122A-122C can comprise various light emitters configured to emit visible light to aid imaging device 120. In examples, lighting devices 122A-122C can comprise light emitting diodes (LEDs). In examples, the diameter of imaging device 120 can be in the range of 0.7 mm to approximately 0.3 mm. In examples, the diameter of each of lighting devices 122A-122C can be in the range of 0.7 mm to approximately 0.3 mm. In an example, a 0.5 mm imaging microchip can be surrounded by a ring of light fibers, each having a diameter of approximately 0.3 mm. In examples, a 0.5 mm imaging microchip can be surrounded by a hollow light fiber having a diameter of approximately 0.8 mm. In a particular example, imaging guidewire can have a diameter of approximately 0.8 mm for use with a duodenoscope with a working channel diameter of approximately 4.2 mm such that the total cross-sectional thickness of concentrically mounted accessory 104 and under-mounted accessory is approximately 3.4 mm or less.

FIG. 3 is a schematic diagram of endoscopy system 10 comprising imaging and control system 12 and endoscope 14. The system of FIG. 3 is an illustrative example of an endoscopy system suitable for use with the systems, devices and methods described herein, such as imaging guidewires and accessories configured to be guided thereby. According to some examples, endoscope 14 can be insertable into an anatomical region for imaging and/or to provide passage of one or more sampling devices for biopsies, or one or more therapeutic devices for treatment of a disease state associated with the anatomical region, such as a stent. Endoscope 14 can, in advantageous aspects, interface with and connect to imaging and control system 12. In the illustrated example, endoscope 14 comprises a duodenoscope, though other types of endoscopes can be used with the features and teachings of the present disclosure.

Imaging and control system 12 can comprise control unit 16, output unit 18, input unit 20, light source unit 22, fluid source 24 and suction pump 26.

Imaging and control system 12 can include various ports for coupling with endoscopy system 10. For example, control unit 16 can include a data input/output port for receiving data from and communicating data to endoscope 14. Light source unit 22 can include an output port for transmitting light to endoscope 14, such as via a fiber optic link. Fluid source 24 can include a port for transmitting fluid to endoscope 14. Fluid source 24 can comprise a pump and a tank of fluid or can be connected to an external tank, vessel or storage unit. Suction pump 26 can comprise a port used to draw a vacuum from endoscope 14 to generate suction, such as for withdrawing fluid from the anatomical region into which endoscope 14 is inserted. Output unit 18 and input unit 20 can be used by an operator of endoscopy system 10 to control functions of endoscopy system 10 and view output of endoscope 14. Control unit 16 can additionally be used to generate signals or other outputs from treating the anatomical region into which endoscope 14 is inserted. In examples, control unit 16 can generate electrical output, acoustic output, a fluid output and the like for treating the anatomical region with, for example, cauterizing, cutting, freezing and the like.

Endoscope 14 can comprise insertion section 28, functional section 30 and handle section 32, which can be coupled to cable section 34 and coupler section 36. Coupler section 36 can be connected to control unit 16 to connect to endoscope 14 to multiple features of control unit 16, such as input unit 20, light source unit 22, fluid source 24 and suction pump 26.

Insertion section 28 can extend distally from handle section 32 and cable section 34 can extend proximally from handle section 32. Insertion section 28 can be elongate and include a bending section, and a distal end to which functional section 30 can be attached. The bending section can be controllable (e.g., by control knob 38 on handle section 32) to maneuver the distal end through tortuous anatomical passageways (e.g., stomach, duodenum, kidney, ureter, etc.). Insertion section 28 can also include one or more working channels (e.g., an internal lumen) that can be elongate and support insertion of one or more therapeutic tools of functional section 30, such as modular scope system 100 of FIG. 1. The working channel can extend between handle section 32 and functional section 30. Additional functionalities, such as fluid passages, guide wires, and pull wires can also be provided by insertion section 28 (e.g., via suction or irrigation passageways, and the like).

Handle section 32 can comprise knob 38 as well as port 40A. Knob 38 can be coupled to a pull wire, or other actuation mechanisms, extending through insertion section 28. Port 40A, as well as port 40B (FIG. 2) can be configured to couple various electrical cables, guide wires, auxiliary scopes, tissue collection devices of the present disclosure, fluid tubes and the like to handle section 32 for coupling with insertion section 28.

Imaging and control system 12, according to examples, can be provided on a mobile platform (e.g., cart 41) with shelves for housing light source unit 22, suction pump 26, image processing unit 42 (FIG. 4), etc. Alternatively, several components of imaging and control system 12 shown in FIGS. 3 and 4 can be provided directly on endoscope 14 so as to make the endoscope “self-contained.”

Functional section 30 can comprise components for treating and diagnosing anatomy of a patient. Functional section 30 can comprise an imaging device, an illumination device and an elevator, such as is described further with reference to elevator 54 of FIGS. 5A-5C. Furthermore, functional section 30 can comprise one or more electrodes conductively connected to handle section 32 and functionally connected to imaging and control system 12 to perform ablation and the like. Similarly, functional section 30 can be configured to perform cauterizing, cutting, freezing and the like. In additional examples, functional section 30 can incorporate tissue collectors or tissue retrieval devices to withdraw biological matter from the anatomy.

FIG. 4 is a schematic diagram of endoscopy system 10 of FIG. 3 comprising imaging and control system 12 and endoscope 14. FIG. 4 schematically illustrates components of imaging and control system 12 coupled to endoscope 14, which in the illustrated example comprises a duodenoscope. Imaging and control system 12 can comprise control unit 16, which can include or be coupled to image processing unit 42, treatment generator 44 and drive unit 46, as well as light source unit 22, input unit 20 and output unit 18. Coupler section 36 can be connected to control unit 16 to connect to endoscope 14 to multiple features of control unit 16, such as image processing unit 42 and treatment generator 44. In examples, port 40A can be used to insert another instrument or device, such as a daughter scope or auxiliary scope, into endoscope 14. Such instruments and devices can be independently connected to control unit 16 via cable 47. In examples, port 40B can be used to connect coupler section 36 to various inputs and outputs, such as video, air, light and electric. Control unit 16 can be in communication with modular scope system 100 and can be configured to operate features thereof in conjunction with control devices 110, 132 and 142 (FIG. 1), as well as to provide various inputs to modular scope system 100, such as power, ablation energy, cauterizing energy, laser energy, irrigation fluids, suction, compressed gas and the like. Control unit 16 can be configured to activate a camera to view target tissue distal of endoscope 14. Likewise, control unit 16 can be configured to activate light source unit 22 to shine light on surgical instruments extending from endoscope 14. In examples, endoscope 14 can comprise a duodenoscope into which imaging guidewire 102, accessory 104 and accessory 106 can be inserted.

Image processing unit 42 and light source unit 22 can each interface with endoscope 14 (e.g., at functional unit 30) by wired or wireless electrical connections. Imaging and control system 12 can accordingly illuminate an anatomical region, collect signals representing the anatomical region, process signals representing the anatomical region, and display images representing the anatomical region on display unit 18. Imaging and control system 12 can include light source unit 22 to illuminate the anatomical region using light of desired spectrum (e.g., broadband white light, narrow-band imaging using preferred electromagnetic wavelengths, and the like). Imaging and control system 12 can connect (e.g., via an endoscope connector) to endoscope 14 for signal transmission (e.g., light output from light source, video signals from imaging system in the distal end, diagnostic and sensor signals from a diagnostic device, and the like).

Fluid source 24 (FIG. 3) can be in communication with control unit 16 and can comprise one or more sources of air, saline or other fluids, as well as associated fluid pathways (e.g., air channels, irrigation channels, suction channels) and connectors (barb fittings, fluid seals, valves and the like). Imaging and control system 12 can also include drive unit 46, which can be an optional component. Drive unit 46 can comprise a motorized drive for advancing a distal section of endoscope 14, as described in at least PCT Pub. No. WO 2011/140118 A1 to Frassica et al., titled “Rotate-to-Advance Catheterization System,” which is hereby incorporated in its entirety by this reference.

FIGS. 5A-5C illustrate a first example of functional section 30 of endoscope 14 of FIGS. 3 and 4. FIG. 5A illustrates a top view of functional section 30 and FIG. 5B illustrates a cross-sectional view of functional section 30 taken along section plane 5B-5B of FIG. 5A. FIGS. 5A and 5B each illustrate “side-viewing endoscope” (e.g., duodenoscope) camera module 50. In side-viewing endoscope camera module 50, illumination and imaging systems are positioned such that the viewing angle of the imaging system corresponds to a target anatomy lateral to central longitudinal axis A1 of endoscope 14.

In the example of FIGS. 5A and 5B, side-viewing endoscope camera module 50 can comprise housing 52, elevator 54, fluid outlet 56, illumination lens 58 and objective lens 60. Housing 52 can form a fluid tight coupling with insertion section 28. Housing 52 can comprise opening for elevator 54. Elevator 54 can comprise a mechanism for moving a device inserted through insertion section 28. In particular, elevator 54 can comprise a device that can bend an elongate device extended through insertion section 28 along axis A1, as is discussed in greater detail with reference to FIG. 5C. Elevator 54 can be used to bend the elongate device at an angle to axis A1 to thereby treat the anatomical region adjacent side-viewing endoscope camera module 50. Elevator 54 is located alongside, e.g., radially outward of axis A1, illumination lens 58 and objective lens 60.

As can be seen in FIG. 5B, insertion section 28 can comprise central lumen 62 through which various components can be extended to connect functional section 30 with handle section 32 (FIG. 4). For example, illumination lens 58 can be connected to light transmitter 64, which can comprise a fiber optic cable or cable bundle extending to light source unit 22 (FIG. 3). Likewise, objective lens 60 can be coupled to prism 66 and imaging unit 67, which can be coupled to wiring 68. Also, fluid outlet 56 can be coupled to fluid line 69, which can comprise a tube extending to fluid source 24 (FIG. 3). Other elongate elements, e.g., tubes, wires, cables, can extend through lumen 62 to connect functional section 30 with components of endoscopy system 10, such as suction pump 26 (FIG. 3) and treatment generator 44 (FIG. 4).

FIG. 5C a schematic cross-sectional view taken along section plane 5C-5C of FIG. 5A showing an elevator 54. Elevator 54 can comprise deflector 55 that can be disposed in space 53 of housing 52. Deflector 55 can be connected to wire 57, which can extend through tube 59 to connect to handle section 32. Wire 57 can be actuated, such as by rotating a knob, pulling a lever, or pushing a button on handle section 32. Movement of wire 57 can cause rotation, e.g., clockwise, from a first position of deflector 55 about pin 61 to a second position of deflector 55, indicated by 55′. Deflector 55 can be actuated by wire 57 to move the distal portion of instrument 63 extending through window 65 in housing 52.

Housing 52 can comprise accommodation space 53 that houses deflector 55. Instrument 63 can comprise forceps, a catheter, or the like that extends through lumen 62. A proximal end of deflector 55 can be attached to housing 52 at pin 61 8 provided to the rigid tip 21. A distal end of deflector 55 can be located below window 65 within housing 52 when deflector 55 is in the lowered, or un-actuated, state. The distal end of deflector 55 can at least partially extend out of window 65 when deflector 55 is raised, or actuated, by wire 57. Instrument 63 can slide on angled ramp surface 51 of deflector 55 to initially deflect the distal end of instrument 63 toward window 65. Angled ramp surface 51 can facilitate extension of the distal portion of instrument 63 extending from window 65 at a first angle relative to the axis of lumen 62. Angled ramp surface 51 can include groove 69, e.g. a v-notch, to receive and guide instrument 63. Deflector 55 can be actuated to bend instrument 63 at a second angle relative to the axis of lumen 62, which is closer to perpendicular that the first angle. When wire 57 is released, deflector 55 can be rotated, e.g., counter-clockwise, back to the lowered position, either by pushing or relaxing of wire 57.

FIG. 6 is a diagram illustrating endoscope 14 and imaging guidewire 102 inserted into anatomy 180 to reach duodenum D. Endoscope 14 can extend into the mouth, through the esophagus, through the stomach of a patient to reach duodenum D.

Endoscope 14 can comprise functional module 50 and shaft 34 and can be connected to control unit 16. Coupler section 36 of endoscope 14 can be connected to control unit 16. Control unit 16 can include other components, including light source unit 22, image processing unit 42 and treatment generator 44, as is described with reference to endoscopy system 10 (FIG. 3) and control unit 16 (FIG. 4). Additionally, control unit 16 can comprise control, activation, energization, lighting and imaging components, as well as others, for operating modular scope system 100 as described herein.

Duodenum D can comprise common bile duct 182, duct wall 184, sphincter of Oddi 186, and main pancreatic duct 188. Duodenum D comprises an upper part of the small intestine. Common bile duct 182 carries bile from the gallbladder and liver (not illustrated) and empties the bile into the duodenum D through sphincter of Oddi 186. Main pancreatic duct 188 carries pancreatic juice from the exocrine pancreas (not illustrated) to common bile duct 182. Sometimes it can be desirable to remove biological matter, e.g., tissue, from bile duct 182 or main pancreatic duct 188 to analyze the tissue to, for example, diagnose diseases or maladies of the patient such as cancer.

Functional module 50 can comprise elevator 54. Endoscope 14 can further comprise lumen 62 into which imaging guidewire 102 can be inserted. Imaging guidewire 102 can comprise imaging device 120. Though not shown for simplicity, imaging guidewire 102 can itself include functional components, such as lighting devices 122A, 122B and 122C, to facilitate navigation of imaging guidewire 102 from endoscope 14 through anatomy 180 and to facilitate viewing of components extending from imaging guidewire 102. Elevator 54 of integrated steering capabilities of imaging guidewire 102, e.g., pull wires, can be used to turn imaging guidewire 102 from lumen 62 toward sphincter of Oddi 186.

In certain duodenoscopy procedures (e.g., Endoscopic Retrograde Cholangio-Pancreatography, hereinafter “ERCP” procedures) an auxiliary scope (also referred to as daughter scope, or cholangioscope) can be attached and advanced through a central lumen (e.g., lumen 62) of the “main scope” (also referred to as mother scope, or duodenoscope), such as endoscope 14. However, insertion of the daughter scope into the mother scope can limit the procedures performed thereafter without having to remove the daughter scope and insert another instrument. This process can be time consuming as it can involve having to renegotiate entry into sphincter of Oddi 186. As discussed in greater detail below, imaging guidewire 102 can be guided into sphincter of Oddi 186. Therefrom, a surgeon that is operating imaging guidewire 102 can navigate imaging guidewire 102 through lumen 62 toward the gall bladder, liver or other locations in the gastrointestinal system to evaluate the anatomy and determine which instrument is desired to perform various procedures. The surgeon can navigate imaging guidewire 102 past entry 190 of common bile duct 182 and into passage 192 of common bile duct 182, or into entry 190. Imaging guidewire 102 can be used to guide concentrically mounted accessory 104 and under-mounted accessory 106 to the anatomy to perform various procedures, such as implanting a stent and obtaining biological matter, such as by sliding along imaging guidewire 102. The accessory devices can have their own functional devices, such as a light source, accessories, and biopsy channel, for therapeutic procedures. As described with reference to FIGS. 9 and 10, the accessory devices can include various features for gathering biological matter, such as tissue. The biological matter can then be removed from the patient, typically by removal of the additional device from the auxiliary device, so that the removed biological matter can be analyzed to diagnose one or more conditions of the patient. According to several examples, endoscope 14 and the devices inserted therein can be suitable for the removal of cancerous or pre-cancerous matter (e.g., carcinoma, sarcoma, myeloma, leukemia, lymphoma and the like), endometriosis evaluation, biliary ductal biopsies, and the like.

FIG. 7A is a schematic view of accessory device 200 coupled with imaging guidewire 102. Accessory device 200 can comprise stent 202 and delivery device 204. Accessory device 200 can comprise an example of concentrically mounted accessory 104. Stent 202 can comprise inflation balloon 206 and expandable body 208. FIG. 7A shows expandable body 208 and inflation balloon 206 in a collapsed state. Expandable body 208 can comprise a mesh body having outer diameter 210 and internal space 212. Delivery device 204 can extend distally toward stent 202, through expandable body 208 and can extend distally from stent 202. Inflation balloon 206 can comprise an inflatable bladder having internal space 214.

In examples, expandable body 208 and balloon 206 can be navigated to duodenum D using the various devices described herein, such as imaging guidewire 102 and endoscope 14. Delivery device 204 can comprise an insertion instrument, tube or sheath that can be used to extend stent 202 through the working channel of a scope, such as lumen 62 of endoscope 14, while be positioned around imaging guidewire 102.

FIG. 7B is a schematic view of accessory device 200 of FIG. 7A in an expanded state. Balloon 206 can be inflated to enlarge expandable body 208 from diameter D1s to diameter D2s. Balloon 206 can be inflated by the passing of pressurized air or another gas through delivery device 204 or a tube therein. Balloon 206 can thereby expand internal space 212. Material of expandable body 208 can stretch or deform to enlarge to the expanded state. Material of expandable body 208 can maintain shape after balloon 206 is deflated. As such internal space 212 can be maintained at diameter D2s. Thus, balloon 206 and delivery device 204 can be withdrawn from stent 202 and the patient through the working channel of the insertion device.

FIG. 7C is a schematic view of duodenum D of FIG. 6 with expandable body 208 of stent 202 inserted into duodenal papilla 230. Expandable body 208 of stent 202 can comprise an annular cylindrical body that pushes sphincter of Oddi 186 into an enlarged state. Expandable body 208 can be delivered to duodenum D in a collapsed state and then enlarged to provide a portal into common bile duct 182.

In order to push stent 202 into duodenal papilla 230, sphincter of Oddi 186 (FIG. 6) can be cut to relax the tissue of duodenal papilla 230 to facilitate insertion of stent 202. Duodenal papilla 230 can be cauterized to reach sphincter of Oddi 186. As such, duodenal papilla 230 can be opened or enlarged to accept stent 530.

FIG. 8 is a close-up schematic view of attachment mechanism 170 comprising a rail system used to connect under-mounted accessory 106 of FIGS. 1 and 2B, wherein under-mounted accessory comprises working channel 160, irrigation channel 162 and auxiliary channel 164. In the example of FIG. 8, guidewire component 172 of attachment mechanism 170 can comprise slot 250 and accessory component 174 of attachment mechanism 170 can comprise rail 252. Slot 250 can comprise base 254 and opening 256. Rail 252 can comprise head 258 and neck 260. In examples, rail 252 can be provided on imaging guidewire and slot 250 can be provided on under-mounted accessory 106.

Slot 250 and rail 252 can be configured to allow accessory 106 to attach to imaging guidewire 102 in a slidable manner. Slot 250 and rail 252 can interact to prevent circumferential and radial movement of accessory 106 relative to imaging guidewire 102, but to allow axial movement. Head 258 and base 254 are illustrated as having oblong or capsule shapes. However, other shapes can be used, such as circular, rectilinear and arcuate. In examples, the shapes of head 258 and base 254 can provide radial interference to movement of accessory 106 away from imaging guidewire 102. Thus, head 258 can be wider than opening 256 to prevent accessory 106 from moving radially away from imaging guidewire. Head 258 can be slightly smaller than base 254 to allow accessory 106 to move freely along imaging guidewire 102.

FIG. 9 is a schematic view of modular scope system 100 comprising imaging guidewire 102 attached to under-mounted accessory 106A having irrigation channel 270 and laser fiber 272. Under-mounted accessory 106A can comprise an example configuration of accessory 106 of FIGS. 1-2B. Irrigation channel 270 can be configured to dispense irrigation fluid 274. Laser fiber 272 can be configured to emit light beam 276. The distal end of modular scope system 100 can be inserted into anatomic duct 280 where biological material 282 can be located. In examples, biological material 282 can comprise a stone, such as a kidney stone or a gallstone.

Laser fiber 272 can be integrated into material, e.g., embedded therein, of under-mounted accessory 106A. Laser fiber 272 can be connected to control device 142 of accessory 106A so that a user can selectively emit light beam 276. Irrigation channel 270 can be connected to control device 142 and control unit 16, where a source of irrigation fluid can be supplied.

As discussed herein, imaging guidewire 102 can be navigated to anatomic duct 280 using viewing module 119 without accessory 106A attached thereto. Imaging and viewing light of viewing module 119 can be used to view anatomic duct 280 before accessory 106A is attached to imaging guidewire 102 in order to assess the anatomy. A surgeon can review the video images to determine the presence, location and condition of biological material 282. In the example of FIG. 9, a surgeon can decide that an accessory incorporating laser lithotripsy capabilities is desired to treat biological material 282. As such, attachment mechanism 170 (FIG. 2B) can be utilized to couple accessory 106A to imaging guidewire 102. Attachment mechanism 170 is not illustrated in FIG. 9 for clarity. However, attachment mechanism 170 can comprise stop 290 that can be used to limit the distance that accessory 106A can be extended along imaging guidewire 102, as discussed below.

Shaft 138 of accessory 106A can have distal end face 284 wherefrom irrigation fluid 274 and light beam 276 can be emitted. For example, a distal end of laser fiber 272 can terminate at or near distal end face 284. Likewise, irrigation channel 270 can open at distal end face 284. Thus, irrigation fluid 274 and light beam 276 can be emitted distally from shaft 138 in the direction of biological material 282.

Laser beam 276 can be configured to break apart biological material 282 into smaller pieces to facilitate disposal. In examples, broken-up pieces of biological material 282 can be processed naturally by the anatomy, such as by being dissolved or passed through the gastrointestinal tract. In examples, irrigation fluid 274 can be dispensed before, during and after light beam 276 is used to wash away broken-up pieces of biological material 282 to facilitate disposal. Additionally, irrigation fluid 274 can be used to clear debris from viewing module 119.

In additional examples, accessory 106A can include a working channel wherein a tissue removal device, such as a basket, can be inserted for removal of broken-up pieces of biological material 282. In examples, laser fiber 272 can be configured to operate similarly as a laser lithotripter. Stop 290 can be used to limit how close end face 284 can come to viewing module 119. This can be used by a surgeon to obtain consistent application of light beam 276 to biological material 282. Additionally, biological material 282 can become broken up by shockwave 292 caused by impact of light beam 276 with biological material 282. Stop 290 can be used to limit how close laser fiber 272 can come to shockwave 292, thereby preventing or inhibiting damage to laser fiber 272.

FIG. 10 is a schematic view of modular scope system 100 comprising imaging guidewire 102 attached to accessory 106B having fluid channel 300 and working channel 302 into which is inserted removal device 304. Accessory 106B can comprise an example configuration of accessory 106 of FIGS. 1-2B. Fluid channel 300 can be configured to dispense irrigation fluid 306. Removal device 304 can comprise jaws 308, shaft 310 and controller 312.

The distal end of modular scope system 100 can be inserted into anatomic duct 320 where biological material 322 can be located. In examples, biological material 322 can comprise a stone, such as a kidney stone or a gallstone.

As discussed herein, imaging guidewire 102 can be navigated to anatomic duct 320 using viewing module 119 without accessory 106B attached thereto. Imaging and viewing light of viewing module 119 can be used to view anatomic duct 320 before accessory 106B is attached to imaging guidewire 102 in order to assess the anatomy. A surgeon can review the video images to determine the presence, location and condition of biological material 322. In the example of FIG. 10, a surgeon can decide that an accessory incorporating tissue removal capabilities is desired to treat biological material 322. For example, stones comprising biological material 322 may be small enough to be removed through the anatomy without requiring being broken up. As such, attachment mechanism 170 (FIG. 2B) can be utilized to couple accessory 106B to imaging guidewire 102. Attachment mechanism 170 is not illustrated in FIG. 10 for clarity. However, attachment mechanism 170 can comprise stop 324 that can be used to limit the distance that accessory 106B can be extended along imaging guidewire 102, as discussed below.

Shaft 138 of accessory 106B can have distal end face 326 wherefrom irrigation fluid 306 and removal device 304 can be emitted. For example, jaws 308 of removal device 304 can be extended beyond distal end face 326 by shaft 310. Likewise, irrigation channel 300 can open at distal end face 326. Thus, fluid 306 and jaws 308 can be emitted distally from shaft 138 in the direction of biological material 322.

Removal device 304 can be configured as any suitable device configured to obtain tissue samples from within a patient. Removal device 304 can additionally comprise a component or device for interacting with a patient, such as those configured to cut, slice, pull, saw, punch, twist or auger tissue, and the like. Specifically, removal device 304 can comprise any device suitable for removing tissue from a patient, such as a blade, punch or an auger. Removal device 304 can be configured to physically separate portions of tissue of a patient from other larger portions of tissue in the patient. In additional examples, removal device 304 can be configured to simply collect biological matter from the patient that does not need physical separation, such as mucus or fluid. In the illustrated example, removal device 304 can comprise forceps where jaws 308 can be configured as sharpened or serrated jaws pivotably connected at a hinge. Removal device 304 can, however, be configured as a variety of devices capable of collecting biological matter, such as a punch, an auger, a blade, a saw and the like, as mentioned. Removal device 304 can alternatively or additionally comprise a biological matter collection device, a biological matter retrieval device, a tissue collection device and tissue retrieval device.

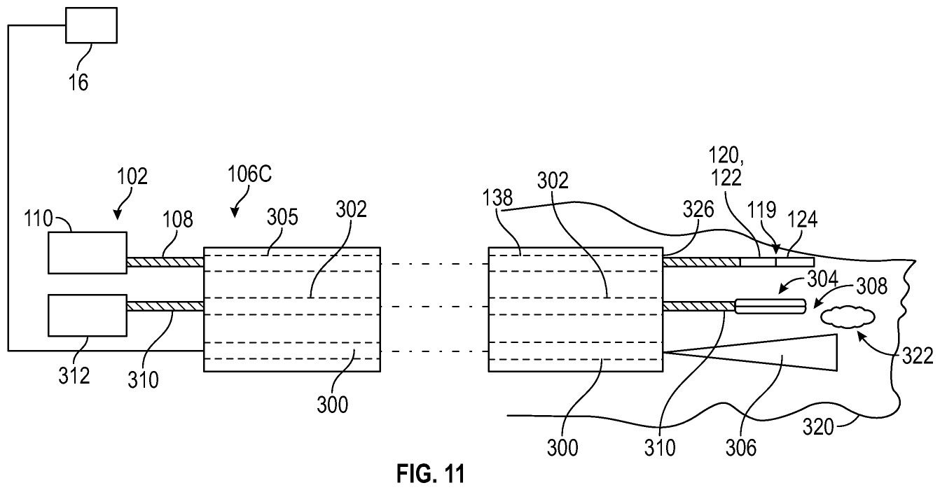

FIG. 11 is a schematic view of modular scope system 100 comprising imaging guidewire 102 extending through accessory 106C having fluid channel 300, working channel 302 and guidewire channel 305. Removal device 304 can be inserted into working channel 302. Imaging guidewire 102 can be inserted into guidewire channel 305. Accessory 106C can comprise an example configuration of accessory 106 of FIGS. 1-2B. Fluid channel 300 can be configured to dispense irrigation fluid 306. Removal device 304 can comprise jaws 308, shaft 310 and controller 312.

The distal end of modular scope system 100 can be inserted into anatomic duct 320 where biological material 322 can be located. In examples, biological material 322 can comprise a stone, such as a kidney stone or a gallstone.

Accessory 106C can be configured similarly as accessory 106B of FIG. 10 except guidewire channel 305 can be incorporated into shaft 138 and any devices or capabilities for attaching imaging guidewire 102 to the exterior of shaft 138 can be omitted. As such, accessory 106C can comprise an over-the-wire mounted accessory similar to concentrically mounted accessory 104 in that imaging guidewire 102 is within the accessory in both examples and not on the exterior of the accessory.

FIGS. 9-11 illustrate particular configurations of accessories 106A, 106B and 106C that can be used as accessory housings in conjunction with imaging guidewire 102. The particular features of other accessories and housings to be used with imaging guidewire 102 can be selected to meet different needs for different surgical procedures or for different surgeon preferences. Thus, accessories 106A, 106B and 106C can be configured to have more or fewer working channels and accessory or auxiliary channels in other configurations and different stone capture or stone fragmenting capabilities.

### EXAMPLES

Example 1 is a modular endoscope system comprising: an imaging guidewire comprising: an elongate shaft extending from a proximal end to a distal end; an imaging device located proximate the distal end of the elongate shaft; and a lighting element located proximate the distal end of the elongate shaft; and an intervention accessory configured to slide along the elongate shaft to provide a medical intervention.

In Example 2, the subject matter of Example 1 optionally includes a cover located at a distal end of the elongate shaft, the cover being tapered to push anatomy away from the imaging device.

In Example 3, the subject matter of Example 2 optionally includes wherein the cover is transparent and is positioned such that the imaging device can view through the cover.

In Example 4, the subject matter of any one or more of Examples 1-3 optionally include wherein the lighting element comprises a plurality of light emitters.

In Example 5, the subject matter of Example 4 optionally includes wherein the lighting element comprises a ring shape and the imaging device is positioned within the ring shape.

In Example 6, the subject matter of any one or more of Examples 1-5 optionally include wherein the elongate shaft has an outer profile shape and the imaging device and the lighting element are located within the outer profile shape.

In Example 7, the subject matter of Example 6 optionally includes an outer profile shape that is circular and a diameter of the outer profile shape in a range of approximately 0.8 mm to approximately 3.0 mm.

In Example 8, the subject matter of Example 7 optionally includes a combined outer diameter of the imaging guidewire and the intervention accessory not exceeding approximately 5.0 mm.

In Example 9, the subject matter of any one or more of Examples 6-8 optionally include a slide feature extending along at least a portion of the elongate shaft between the proximal end and the distal end.

In Example 10, the subject matter of Example 9 optionally includes wherein the slide feature comprises a slot configured to receive a mating rail.

In Example 11, the subject matter of Example 10 optionally includes wherein the slot is located within outer profile shape.

In Example 12, the subject matter of any one or more of Examples 10-11 optionally include wherein the slot comprises a radial catch.

In Example 13, the subject matter of Example 12 optionally includes wherein the slot has a T-shape.

In Example 14, the subject matter of any one or more of Examples 9-13 optionally include wherein the slide feature comprises a distal stop.

In Example 15, the subject matter of any one or more of Examples 9-14 optionally include wherein the intervention accessory is configured to slide over the slide feature disconnected from the slide feature.

In Example 16, the subject matter of Example 15 optionally includes wherein the intervention accessory comprises a stent configured to fit around the elongate shaft.

In Example 17, the subject matter of Example 16 optionally includes wherein the stent comprises in insertion shaft configured to position the stent along the elongate shaft.

In Example 18, the subject matter of any one or more of Examples 9-17 optionally include wherein the intervention accessory is configured to slide along the slide feature connected to the slide feature.

In Example 19, the subject matter of any one or more of Examples 1-18 optionally include wherein the intervention accessory comprises an elongate housing having at least one channel extending at least partially therethrough.

In Example 20, the subject matter of Example 19 optionally includes wherein the elongate housing further comprises a laser fiber extending at least partially therethrough.

In Example 21, the subject matter of any one or more of Examples 19-20 optionally include wherein the elongate housing further comprises an irrigation channel configured to convey a fluid through the elongate housing.

In Example 22, the subject matter of any one or more of Examples 19-21 optionally include a tissue retrieval device configured to extend through the at least one channel.

Example 23 is the modular endoscope system of Example 1, further comprising a duodenoscope having a working channel into which the imaging guidewire and accessory can simultaneously fit.

Example 24 is a method of providing a medical intervention to an internal anatomic location, the method comprising: inserting an imaging guidewire into an anatomic passageway; viewing target anatomy with imaging capabilities of the imaging guidewire; pushing an intervention accessory along the imaging guidewire to the target anatomy; and operating the intervention accessory to provide an intervention on the target anatomy.

In Example 25, the subject matter of Example 24 optionally includes intraoperatively determining an intervention action from viewing the target anatomy with the imaging capabilities of the imaging guidewire.

In Example 26, the subject matter of Example 25 optionally includes wherein the intervention accessory is selected based on a determined intervention action.

In Example 27, the subject matter of Example 26 optionally includes wherein the intervention action comprises opening a sphincter in the internal anatomic location.

In Example 28, the subject matter of Example 27 optionally includes opening the sphincter with a stent comprising the intervention accessory.

In Example 29, the subject matter of any one or more of Examples 24-28 optionally include wherein pushing the intervention accessory along the imaging guidewire to the target anatomy comprises: positioning the intervention accessory around the imaging guidewire.

In Example 30, the subject matter of any one or more of Examples 26-29 optionally include wherein the intervention action comprises displacing biological material.

In Example 31, the subject matter of Example 30 optionally includes breaking-up the biological material with a laser lithotripter comprising the intervention accessory.

In Example 32, the subject matter of any one or more of Examples 30-31 optionally include removing the biological material with a tissue removal device comprising the intervention accessory.

In Example 33, the subject matter of any one or more of Examples 30-32 optionally include irrigating the target anatomy with the intervention accessory.

In Example 34, the subject matter of any one or more of Examples 30-33 optionally include wherein pushing the intervention accessory along the imaging guidewire to the target anatomy comprises: sliding the intervention accessory along a slide feature of the imaging guidewire.

In Example 35, the subject matter of Example 34 optionally includes preventing radial and circumferential displacement of the intervention accessory relative to the imaging guidewire with the slide feature.

In Example 36, the subject matter of any one or more of Examples 34-35 optionally include wherein the slide feature of the imaging guidewire comprises a slot and the intervention accessory comprises a rail configured to mate with the slot.

In Example 37, the subject matter of Example 36 optionally includes wherein the rail and the slot have complementary T-shaped profiles.

In Example 38, the subject matter of any one or more of Examples 34-37 optionally include preventing the intervention accessory from sliding off the imaging guidewire with a stop.

In Example 39, the subject matter of any one or more of Examples 24-38 optionally include wherein inserting the imaging guidewire into the anatomic passageway comprises inserting the imaging guidewire through a working channel of a duodenoscope.

In Example 40, the subject matter of Example 39 optionally includes wherein pushing the intervention accessory along the imaging guidewire to the target anatomy comprises pushing the intervention accessory through the working channel of the duodenoscope.

In Example 41, the subject matter of any one or more of Examples 24-40 optionally include wherein inserting the imaging guidewire into the anatomic passageway comprises pushing a cover of the imaging guidewire through anatomy to shield the imaging capabilities.

In Example 42, the subject matter of any one or more of Examples 24-41 optionally include wherein viewing target anatomy with the imaging capabilities of the imaging guidewire comprises: viewing the target anatomy from a distal end of the imaging guidewire.

In Example 43, the subject matter of any one or more of Examples 24-42 optionally include wherein viewing target anatomy with the imaging capabilities of the imaging guidewire comprises: illuminating the target anatomy with a lighting device; and capturing images of the target anatomy with a camera device.

Each of these non-limiting examples can stand on its own, or can be combined in various permutations or combinations with one or more of the other examples.

### Various Notes

The above detailed description includes references to the accompanying drawings, which form a part of the detailed description. The drawings show, by way of illustration, specific embodiments in which the invention can be practiced. These embodiments are also referred to herein as “examples.” Such examples can include elements in addition to those shown or described. However, the present inventor also contemplates examples in which only those elements shown or described are provided. Moreover, the present inventor also contemplates examples using any combination or permutation of those elements shown or described (or one or more aspects thereof), either with respect to a particular example (or one or more aspects thereof), or with respect to other examples (or one or more aspects thereof) shown or described herein.

In the event of inconsistent usages between this document and any documents so incorporated by reference, the usage in this document controls.

In this document, the terms “a” or “an” are used, as is common in patent documents, to include one or more than one, independent of any other instances or usages of “at least one” or “one or more.” In this document, the term “or” is used to refer to a nonexclusive or, such that “A or B” includes “A but not B,” “B but not A,” and “A and B,” unless otherwise indicated. In this document, the terms “including” and “in which” are used as the plain-English equivalents of the respective terms “comprising” and “wherein.” Also, in the following claims, the terms “including” and “comprising” are open-ended, that is, a system, device, article, composition, formulation, or process that includes elements in addition to those listed after such a term in a claim are still deemed to fall within the scope of that claim. Moreover, in the following claims, the terms “first,” “second,” and “third,” etc. are used merely as labels, and are not intended to impose numerical requirements on their objects.

Method examples described herein can be machine or computer-implemented at least in part. Some examples can include a computer-readable medium or machine-readable medium encoded with instructions operable to configure an electronic device to perform methods as described in the above examples. An implementation of such methods can include code, such as microcode, assembly language code, a higher-level language code, or the like. Such code can include computer readable instructions for performing various methods. The code may form portions of computer program products. Further, in an example, the code can be tangibly stored on one or more volatile, non-transitory, or non-volatile tangible computer-readable media, such as during execution or at other times. Examples of these tangible computer-readable media can include, but are not limited to, hard disks, removable magnetic disks, removable optical disks (e.g., compact disks and digital video disks), magnetic cassettes, memory cards or sticks, random access memories (RAMs), read only memories (ROMs), and the like.

The above description is intended to be illustrative, and not restrictive. For example, the above-described examples (or one or more aspects thereof) may be used in combination with each other. Other embodiments can be used, such as by one of ordinary skill in the art upon reviewing the above description. The Abstract is provided to allow the reader to quickly ascertain the nature of the technical disclosure. It is submitted with the understanding that it will not be used to interpret or limit the scope or meaning of the claims. Also, in the above Detailed Description, various features may be grouped together to streamline the disclosure. This should not be interpreted as intending that an unclaimed disclosed feature is essential to any claim. Rather, inventive subject matter may lie in less than all features of a particular disclosed embodiment. Thus, the following claims are hereby incorporated into the Detailed Description as examples or embodiments, with each claim standing on its own as a separate embodiment, and it is contemplated that such embodiments can be combined with each other in various combinations or permutations. The scope of the invention should be determined with reference to the appended claims, along with the full scope of equivalents to which such claims are entitled.

1. A modular endoscope system comprising:
an imaging guidewire comprising:
an elongate shaft extending from a proximal end to a distal end;
an imaging device located proximate the distal end of the elongate shaft; and
a lighting element located proximate the distal end of the elongate shaft, wherein the lighting element is arranged in an arcuate configuration to at least partially surround the imaging device; and
an intervention accessory configured to slide over the elongate shaft to provide a medical intervention, wherein the intervention accessory comprises an elongate housing having at least one channel extending at least partially therethrough.

2. The modular endoscope system of claim 1, further comprising a cover located at a distal end of the elongate shaft, wherein the cover is tapered to push anatomy away from the imaging device and is transparent such that the imaging device can view through the cover.

3. The modular endoscope system of claim 1, wherein the lighting element comprises a hollow light fiber surrounding the imaging device.

4. The modular endoscope system of claim 1, wherein the lighting element comprises a plurality of light emitters.

5. The modular endoscope system of claim 4, wherein the lighting element comprises a plurality of light fibers arranged in a ring shape and the imaging device comprises an imaging microchip that is positioned within the ring shape.

6. The modular endoscope system of claim 1, wherein the elongate shaft has an outer profile shape and the imaging device and the lighting element are located within the outer profile shape.

7. The modular endoscope system of claim 6, wherein:
the outer profile shape is circular and a diameter of the outer profile shape is in a range of approximately 0.8 mm to approximately 3.0 mm; and
a combined outer diameter of the imaging guidewire and the intervention accessory does not exceed approximately 5.0 mm.

8. (canceled)

9. The modular endoscope system of claim 6, further comprising a slide feature extending along at least a portion of the elongate shaft between the proximal end and the distal end.

10. The modular endoscope system of claim 9, wherein the slide feature comprises a slot configured to receive a mating rail, wherein the slot comprises a radial catch.

11. The modular endoscope system of claim 10, wherein the slot is located within the outer profile shape.

12. (canceled)

13. The modular endoscope system of claim 11, wherein the slot has a T-shape.

14. The modular endoscope system of claim 9, wherein the slide feature comprises a distal stop.

15. (canceled)

16. The modular endoscope system of claim 9, wherein the intervention accessory comprises:
a stent configured to fit around the elongate shaft so as to slide over the slide feature disconnected form the slide feature; and
an insertion shaft comprising the elongate housing and that is configured to position the stent along the elongate shaft.

17. The modular endoscope system of claim 1, wherein the intervention accessory comprises one or more pull wires extending through the elongate housing.

18. The modular endoscope system of claim 9, further comprising a second intervention accessory that is configured to slide along the slide feature connected to the slide feature.

19. The modular endoscope system of claim 1, wherein the at least one channel extends through the elongate housing offset from a center axis of the elongate housing.

20. The modular endoscope system of claim 1, wherein the elongate housing further comprises a laser fiber extending at least partially therethrough.

21. The modular endoscope system of claim 1, wherein the elongate housing further comprises an irrigation channel configured to convey a fluid through the elongate housing.

22. The modular endoscope system of claim 1, further comprising a tissue retrieval device configured to extend through the at least one channel.

23. The modular endoscope system of claim 1, further comprising a duodenoscope having a working channel into which the imaging guidewire and accessory can simultaneously fit.

24.-43. (canceled)
